(19) United States
(12) Patent Application Publication
(10) Pub. No.: US 2024/0173338 A1
(43) Pub. Date: May 30, 2024

(54) PREPARATION AND ACTIVITY CHARACTERIZATION OF CANNABICITRAN ENANTIOMERS

(71) Applicant: University of North Carolina at Wilmington, Wilmington, NC (US)

(72) Inventors: Robert Thomas Williamson, Wilmington, NC (US); Jeremy B. Morgan, Wilmington, NC (US); Jared S. Wood, Wilmington, NC (US); William H. Gordon, Wilmington, NC (US)

(73) Assignee: University of North Carolina at Wilmington, Wilmington, NC (US)

(21) Appl. No.: 18/516,608

(22) Filed: Nov. 21, 2023

Related U.S. Application Data
(60) Provisional application No. 63/426,973, filed on Nov. 21, 2022.

(51) Int. Cl. A61K 31/00

(52) U.S. Cl. CPC A61K 31/658

(57) ABSTRACT

In one aspect, compounds and associated pharmaceutical compositions are described herein comprising isolated cannabicitran (CBT-C (3)). In some embodiments, CBT-C (3) enantiomers are isolated and/or prepared. In some further embodiments, the biological activity of enantiomers are determined. In some further embodiments, one or more enantiomers may be incorporated into a composition.

### CROSS-REFERENCE TO RELATED APPLICATIONS

This application claims priority to U.S. Provisional Patent Application No. 63/426,973 filed November 21, 2022, which is incorporated herein by reference in its entirety.

### FIELD

The technology described herein generally relates to the preparation and characterization of enantiomers, more particularly to the isolation and/or purification of cannabicitran and the activity characterization thereof.

### BACKGROUND

Cannabinoids, isolated from the Cannabis sativa plant, have been utilized for medicinal purposes for thousands of years. Several cannabinoids have receptor interactions that indicate the modulation of pain, sleep, and mood disorders, while also exerting beneficial effects in pathological conditions such as inflammation, cancer, addiction, and epilepsy. Cannabinoids such as Δ⁹-tetrahydrocannabinol (Δ⁹-THC) and cannabidiol (CBD) are primarily extracted from Cannabis sativa as single enantiomers, but cis-Δ⁹-THC is known to be scalemic, in contrast, only the (−) enantiomer of trans-CBD is naturally found in the plant.

Determining the absolute configuration of a molecule is a crucial part of a complete molecular characterization, especially in the pursuit of developing effective therapeutics. The infamous story of (+/−)-thalidomide has been of paramount importance to the advancement of modern drug development. (+/−)-Thalidomide was used as a sedative for the treatment of morning sickness in pregnant women but was withdrawn from the market after five years due to the correlation of its use and severe teratogenic effects. Before its official recall, thalidomide had adversely affected approximately 10,000 infants, resulting in limb and bone abnormalities, and almost 50% of the affected infants had died. It was eventually determined that R-(+)-thalidomide produced the desired sedative effects, while S-(−)-thalidomide was responsible for the teratogenic effects. Further, it was determined that the racemization of thalidomide occurs in vitro and even more rapidly in vivo. Thus, enantiomerically pure R-(+)-thalidomide cannot be administered without an immediate partial conversion into S-(−)-thalidomide, the teratogen. The thalidomide tragedy harshly demonstrated the importance of chirality in the context of drug development and stimulated the creation of the FDA. Other chiral pharmaceutical examples like Darvon® (dextropropoxyphene) and Novrad® (levopropoxyphene) provide further illustration of complementary enantiomers targeting completely different receptors and exhibiting unrelated physiological effects. Darvon® elicits analgesic effects, and Novrad® is prescribed as an antitussive.

Recent studies have shown that various secondary metabolites that may be naturally occurring are racemates or scalemic mixtures despite precedence suggesting that they are individual enantiomers. Additionally, previous studies on chiral molecules lack information on enantiomeric purity since they are predominantly concerned with other details such as isolation, biological activity evaluation, and structure elucidation.

Current studies around cannabicitran (CBT-C (3)) indicate a wide range of applications for the cannabinoid, including uses in sunscreen, skin care, headache treatments, and treatment of Alzheimer's disease. Additional studies also revealed that CBT-C (3) reduces intraocular pressure in rabbits. This suggests involvement of the NAGly (GPR18) receptor, a known target of structurally related cannabinoids. Prior research demonstrated that the receptor is linked to modulation of physiopathological processes such as pain, metabolism, and cancer.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards the isolation and/or preparation of enantiomers of (CBT-C, 3) from a cannabinoid extract and further determining their biological activity. Further embodiments are generally directed towards applications using one or more enantiomers or enriched mixtures and/or compositions.

In some instances, a medicament is provided comprising a carrier and an amount of CBT-C. In some instances, the CBT-C is an isolated CBT-C (3) racemate. In some instances, the CBT-C (3) is enriched in (1R,3R,4S) CBT-C (3) enantiomer or enriched in (1S,3S,4R) CBT-C (3) enantiomer. In some instances, the CBT-C is at least one of (1R,3R,4S) CBT-C (3) enantiomer and (1S,3S,4R) CBT-C (3) enantiomer.

In another aspect, methods of isolating and preparing enantiomers of CBT-C, 3 are provided. In some instances, a cannabinoid extract is provided and a CBT-C (3) racemate is isolated. In some instances, at least one of (1R,3R,4S) CBT-C (3) enantiomer and (1S,3S,4R) CBT-C (3) enantiomer is isolated. In some embodiments, the biological activity of at least one of the enantiomers is determined.

These and other embodiments are further described in the following detailed description. It will be appreciated that additional objects, advantages, and novel features of the invention will be set forth in part in the following description.

### BRIEF DESCRIPTION OF THE DRAWINGS

Aspects of the technology presented herein are described in detail below with reference to the accompanying figures, wherein:

FIG. 1 shows structures for the enantiomers of cis-Δ⁹-THC (1), trans-CBD (2), and CBT-C (3), in accordance with some aspects of the present technology;

FIG. 2 depicts truncated geometry-optimized 3D structures for both enantiomers of CBT-C (3), in accordance with some aspects of the present technology;

FIG. 3 depicts an ECD spectrum of a CBT-C (3) reference standard, in accordance with some aspects of the present technology;

FIG. 4 shows ECD spectra of (−)-3 and (+)-3, including TDDFT results derived from the ωB97XD/6-311G++(2d,2p) functional and basis set, in accordance with some aspects of the present technology;

FIG. 5A illustrates analytical SFC chromatograms before and after purification of stereoisomers, in accordance with some aspects of the present technology;

FIG. 5B illustrates analytical SFC chromatograms before and after purification of stereoisomers, in accordance with some aspects of the present technology;

FIG. 5C illustrates analytical SFC chromatograms before and after purification of stereoisomers, in accordance with some aspects of the present technology;

FIG. 6 shows structures for (±)-cis-Δ⁹-THC (1), (±)-trans-CBD (2), (±)-CBT-C (3), CBC* (4), (±)-Δ⁹-trans-THC (5), and CBGA (6), in accordance with some aspects of the present technology;

FIG. 7 illustrates truncated geometry-optimized 3D structures for both enantiomers of CBT-C(3), in accordance with some e aspects of the present technology;

FIG. 8 shows an ECD spectrum of CBT-C(3) obtained from a CBD extract, in accordance with some aspects of the present technology;

FIG. 9A shows UV spectra of (−)-3 and (+)-3, including TDDFT results obtained at the CAM-B3LYP/def2-TZVP level of theory, in accordance with some aspects of the present technology;

FIG. 9B shows ECD spectra of (−)-3 and (+)-3, including TDDFT results obtained at the CAM-B3LYP/def2-TZVP level of theory, in accordance with some aspects of the present technology;

FIG. 10A shows experimental and DFT-predicted VCD spectra for enantiomers of CBT-C (3), in accordance with some aspects of the present technology;

FIG. 10B shows experimental and DFT-predicted VCD spectra for enantiomers of CBT-C (3), in accordance with some aspects of the present technology;

FIG. 11A shows analytical SFC chromatograms for cannabicitran, in accordance with some aspects of the present technology;

FIG. 11B shows analytical SFC chromatograms for cannabicitran, in accordance with some aspects of the present technology;

FIG. 12A shows analytical SFC chromatograms for CBT-C (3) enantiomers, in accordance with some aspects of the present technology;

FIG. 12B shows analytical SFC chromatograms for CBT-C (3) enantiomers, in accordance with some aspects of the present technology;

FIG. 13 shows example ECD data for CBT-C (3), in accordance with some aspects of the present technology;

FIG. 14 illustrates a comparison of predicted ECD data for structures of (−)-(3), in accordance with some aspects of the present technology;

FIG. 15A and 15B illustrate a comparison of experimental and TDDFT-predicted UV and ECD data, in accordance with some aspects of the present technology;

FIG. 16A and 16B illustrate a comparison of experimental and TDDFT-predicted UV and ECD data, in accordance with some aspects of the present technology;

FIG. 17A and 17B illustrates a comparison of experimental and TDDFT-predicted UV and ECD data, in accordance with some aspects of the present technology;

FIG. 18A and FIG. 18B illustrate a comparison of experimental and TDDFT-predicted UV and ECD data, in accordance with some aspects of the present technology;

FIG. 19A and FIB. 19B illustrate a comparison of experimental and TDDFT-predicted UV and ECD data, in accordance with some aspects of the present technology;

FIG. 20A and FIG. 20B illustrate a comparison of experimental and TDDFT-predicted UV and ECD data, in accordance with some aspects of the present technology;

FIG. 21A and 21B illustrate experimental and DFT-predicted IR and VCD spectra of (−)-3, in accordance with some aspects of the present technology;

FIG. 22A and FIG. 22B illustrate experimental and DFT-predicted IR and VCD spectra of (−)-3, in accordance with some aspects of the present technology;

FIG. 23A and FIG. 23B illustrate experimental and DFT-predicted IR and VCD spectra of (+)-3, in accordance with some aspects of the present technology;

FIG. 24A and FIG. 24B illustrate experimental and DFT-predicted IR and VCD spectra of (+)-3, in accordance with some aspects of the present technology;

FIG. 25 shows DFT calculation data for the primary conformer of the methyl-truncated structure of (−)-3, in accordance with some aspects of the present technology; and

FIG. 26 shows DFT calculation data for the primary conformer of the methyl-truncated structure of (+)-3, in accordance with some aspects of the present technology.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10. Further, when the phrase “up to” is used in connection with an amount or quantity; it is to be understood that the amount is at least a detectable amount or quantity. For example, a material present in an amount “up to” a specified amount can be present from a detectable amount and up to and including the specified amount. Additionally, in any disclosed embodiment, the terms “substantially,” “approximately,” and “about” may be substituted with “within [a percentage] of” what is specified, where the percentage includes 0.1, 1, 5, and 10 percent.

Cannabicitran is a cannabinoid that can be found in levels up to ˜10% in commercial “purified” cannabidiol (CBD) extracts. However, few studies have investigated cannabicitran or its origin despite the rapidly increasing interest in the use of cannabinoids for the treatment of a wide range of physiological conditions. Following on a recent detailed NMR and computational characterization of cannabicitran, ECD and TDDFT studies were undertaken to determine the absolute configuration of cannabicitran present in Cannabis sativa extracts. According to aspects of the present technology, it can be determined that cannabicitran product is racemic, raising questions around its presumed enzymatic origin. According to some aspects herein, the isolation and absolute configuration of (−)-cannabicitran and (+)-cannabicitran is provided. Further, several possible scenarios for production of the racemate in the plant and/or during extract processing are provided.

Previous studies have shown that opposite enantiomers of cannabinoids bind to individual receptors with vastly different affinities and oftentimes bind to entirely different protein targets. Enantiomers of cis-Δ⁹-THC (1) and trans-CBD (2) have been shown to exhibit differences in binding affinities towards the CB₁ and CB₂ receptors. For both receptors, (−)-1 has been shown to bind strongly relative to (+)-1. Further, both enantiomers have significant binding affinities towards endocannabinoid degrading enzymes such as MAGL and ABHD6. Unnatural (+)-2 has been shown to bind strongly to CB₁ and CB₂, unlike the natural (−)-2, which is known to bind to other targets such as 5-HT_{1A}, multiple TRPs, and GPR55.

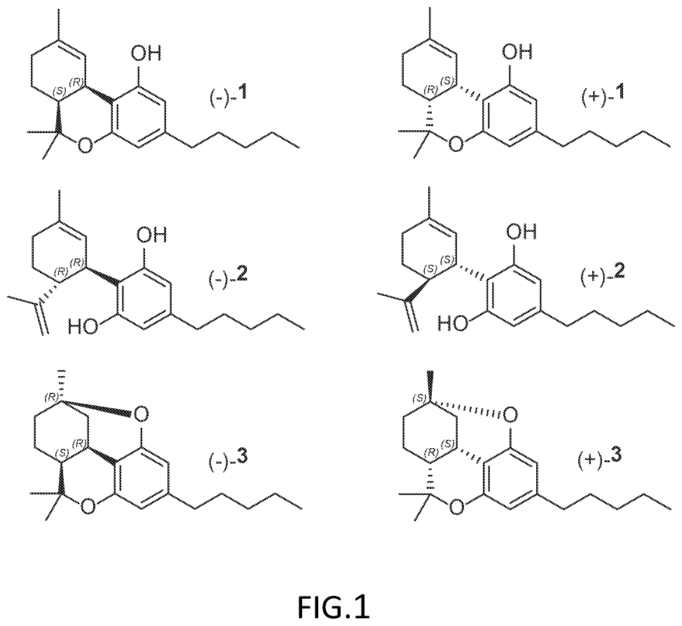

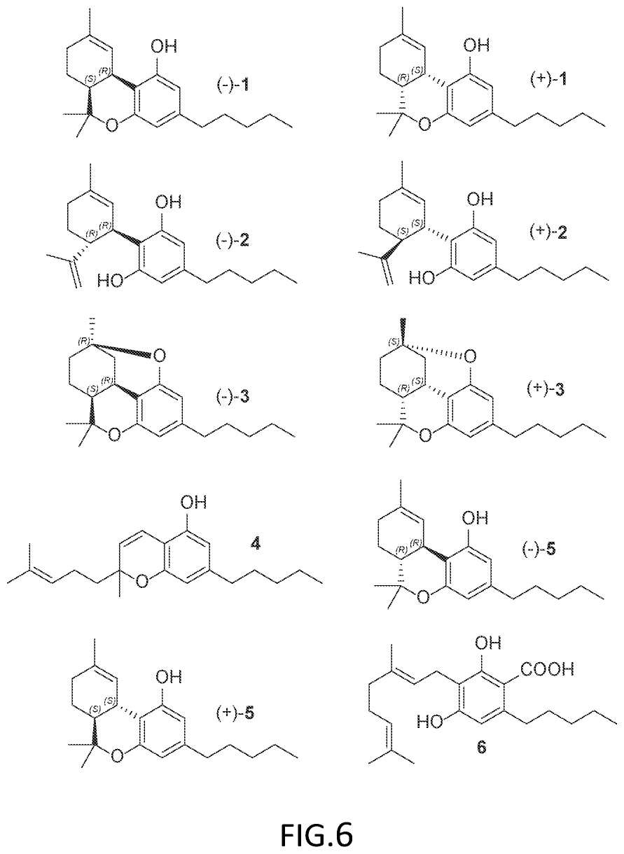

Detailed NMR studies of cannabicitran (CBT-C, 3, FIG. 1, FIG. 6), a previously under-characterized cannabinoid have been published. In one aspect of characterization, to determine the absolute configuration of CBT-C (3) it was determined that it displays no electronic circular dichroism (ECD) signal, which can indicate that CBT-C (3) is racemic. This indication can show that when separation of the enantiomers is achieved using high performance liquid chromatography (HPLC) with a chiral stationary phase. Additional electronic circular dichroism (ECD) experiments performed with the isolated enantiomers provide further evidence that CBT-C (3) can exists as a racemate. Through the utilization of ECD experiments combined with advanced time-dependent density functional theory (TDDFT) calculations and vibrational circular dichroism (VCD) measurements with corresponding DFT computed spectra, the absolute configurations of (−)-3 and (+)-3 can be established.

According to aspects of the present technology, through ECD experimental and TDDFT-predicted data, it can be determined that CBT-C (3) isolated from cannabinoid extracts is racemic, that is, CBT-C (3) can be isolated as a racemate. Further, the CBT-C (3) enantiomers can be resolved by high performance liquid chromatography HPLC, where (−)-3 elutes before (+)-3 with significant resolution. The (−) and (+) enantiomers possess the (1R,3R,4S) and (1S,3S,4R) configurations, respectively. In addition, the ωB97XD/6-311G++(2d,2p) functional and basis set was shown to be the most viable model chemistry for the prediction of ECD data for CBT-C (3).

Generally, embodiments of the technology described herein relate to the purification and/or isolation and/or preparation of cannabicitran enantiomers as well as configuration determinations for the prepared enantiomers. (CBT-C, 3) enantiomers can be prepared and through qPCR techniques demonstrate that the two enantiomers have wide ranging and non-overlapping biological activities. Additionally, in accordance with the present technology, the potency of one or the other of the (CBT-C, 3) enantiomers will be increased as compared to conventional techniques, for example potency may be increased at least two times. According to some aspects, the absolute configuration of cannabicitran present in an extract can be determined. According to some other aspects, the isolation and/or purification and in some instances the absolute configuration of (−)-cannabicitran and (+)-cannabicitran can be achieved and determined. Without intending to be bound by theory, some racemates are known to have synergistic effects which yield higher biological activities relative to their individual enantiomers, and in this context given the extensive collection of chiral metabolites present in Cannabis sativa the preparation and characterization of such racemates. Furthermore, opposite enantiomers of cannabinoids can bind individual receptors with vastly different affinities and oftentimes have high affinity to entirely different protein targets.

Enantiomers of cis-Δ⁹-THC (1) and trans-CBD (2), illustrated in FIG.6, have been shown to exhibit differences in binding affinities towards the CB1 and CB2 receptors. For both receptors, (−)-1 displays stronger binding relative to (+)-1. Further, both enantiomers have significant binding affinities towards endocannabinoid degrading enzymes such as MAGL and ABHD6. Unnatural (+)-2 has been shown to bind strongly to CB₁ and CB₂, unlike the natural (−)-2, which is known to bind to other targets such as 5-HT_{1A}, multiple TRPs, and GPR55.

Various compounds are described herein, as well as the preparation and use thereof. As discussed above, and further illustrated in the examples below, the compounds can exhibit antimicrobial properties in some embodiments, for instance antifungal and antibacterial properties. Some embodiments described herein are further illustrated in the following non-limiting examples.

As will be appreciated, the individual enantiomers that make up the racemic mixture of cannabicitran are biologically and functionally non-equivalent. Historically, many of the drugs currently used in medical practice were marketed as racemates. However, it is well documented that the two enantiomers of a racemate can differ in their pharmacokinetic/pharmacodynamic and efficacy/safety profiles. Often only one of a drug's enantiomers is responsible for the desired physiological and therapeutic effects - while the other enantiomer is less active, inactive or even harmful. Replacing existing racemates with homochiral drugs may result in improved safety and/or efficacy profile of various racemates.

In some aspects, a medicament is provided comprising a carrier and an amount of cannabicitran (CBT-C). As will be appreciated, a carrier can include any suitable carrier compound, ingestible or topical, such as an ointment, oil, gelatin, syrup (e.g. a high viscosity syrup), a confectionary, among other carriers or compounds used for delivering extracts to a user, orally and/or topically. In some instances, the medicament can be a CBT-C that is an isolated CBT-C (3) racemate. In some instances, the CBT-C (3) is enriched in (1R,3R,4S) CBT-C (3) enantiomer, and in some other instances, the CBT-C (3) is enriched in (1S,3S,4R) CBT-C (3) enantiomer. In some further embodiments, the CBT-C is (1R,3R,4S) CBT-C (3) enantiomer, and in some other further embodiments, the the CBT-C is (1S,3S,4R) CBT-C (3) enantiomer. In some even further aspects, the CBT-C is at least one of (+)CBT-C (3) enantiomer and (−)CBT-C (3) enantiomer.

According to some embodiments, methods for purifying and/or isolating molecules and/or compounds or mixtures thereof of a cannabinoid extract are provided. Additionally, determining and/or assigning a configuration or absolute configuration to a purified and/or isolated compound, molecule, or mixture thereof is provided. In some instances the method comprises providing a cannabinoid extract and isolating at least one cannabicitran (CBT-C (3)) racemate. In some instances, the cannabinoid extract is a CBD extract. In some other instances, the cannabinoid extract is a sample of CBT-C (3) obtained from a CBD extract. According to some aspects, the method comprises determining that CBT-C (3) has an ellipticity within 5 degrees of 0 against a wavelength from 180-300 nm. According to some aspects, two CBT-C (3) enantiomers are separated by a chiral-phase column, and wherein the two separated CBT-C (3) enantiomers have equal peak areas, and in some other aspects, a method comprises isolating a first CBT-C (3) enantiomer and a second CBT-C (3) enantiomer based on a chromatographic peak assigned to each of the first enantiomer and the second enantiomer. In some instances, a method comprises determining that the first enantiomer and the second enantiomer have equal and opposite ECD signals. In some further aspects, a method comprises assigning a configuration to each of the first enantiomer and the second enantiomer.

According to some aspects, a method further comprises determining that the first CBT-C (3) enantiomer has a (1R,3R,4S) configuration, and in some other aspects, a method further comprises determining that the second CBT-C (3) enantiomer has a (1S,3S,4R) configuration. According to some other aspects, a method comprises determining that the (1R,3R,4S) CBT-C (3) enantiomer has a specific rotation value between -15 and -25 degrees/(dm g/ml) in methanol at about 589 nm, and/or determining that the(1S,3S,4R) CBT-C (3) enantiomer has a specific rotation value between 15 and 25 degrees/(dm g/ml) in methanol at about 589 nm. According to some even further embodiments, a method comprises validating the configuration to each of the first enantiomer and the second enantiomer based on at least one TDDFT determination at a selected level of theory.

### EXAMPLE 1

### I. CBT-C Isolation and Characterization

Determinations of chiroptical properties can be utilized for the absolute configuration assignment of chiral compounds. According to some aspects, time-dependent density functional theory (TDDFT) determinations of electronic circular dichroism (ECD) and optical rotary dispersion (ORD) can be used to determine the absolute configuration of cannabicitran enantiomers prepared in accordance with some aspects of the present technology.

ECD/ORD Experimental Procedures. UV measurements of CBT-C (3) and the enantiomerically pure (±)-CBT-Cs ((−)-3, (+)-3) in methanol were performed using an Agilent Cary 4000 UV-Vis spectrophotometer. Concentrations were adjusted to provide for A≈0.65 at 213 nm. All ECD experiments were performed on a Chirascan™ circular dichroism spectrometer, using a 1 cm pathlength cell. Molar extinction (Δε) was measured in M⁻¹·cm⁻¹ across a wavelength range of 200-300 nm in increments of 0.5 nm. Optical rotation experiments were performed on a Rudolph Research Analytical Autopol® III Automatic Polarimeter. A 50 mm pathlength cell was used, and concentrations were adjusted to c≈0.25 g/dL.

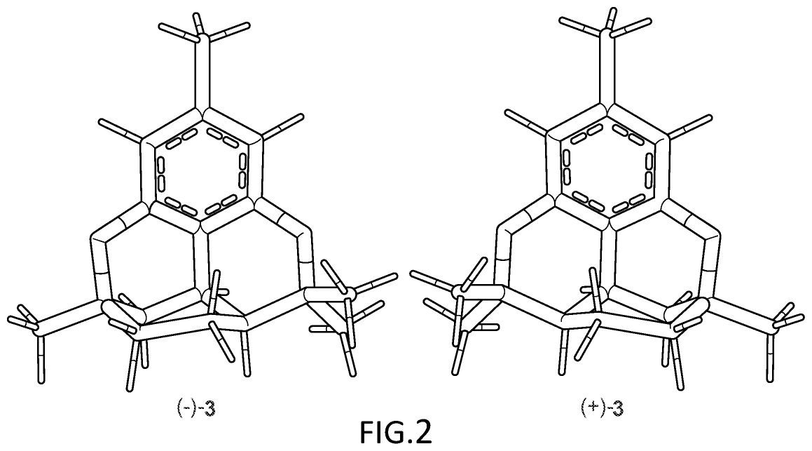

TDDFT Calculations. In a baseline NMR-based study of CBT-C (3), the Schrödinger MacroModel software package was used to perform a Mixed Torsional/Low-Mode Sampling (MTLMOD) conformational search using the OPLS4 force field on a methyl-truncated structure of (−)-3 as is shown in FIG. 2. Geometry optimization was performed on the primary conformer using the B3LYP/6-31G+(d,p) functional and basis set. This optimized structure was used to predict optical rotatory dispersion (ORD) and ECD data using the Gaussian software package through ground state and time-dependent DFT calculations, respectively. The functional and basis sets used for these calculations are shown in Table 1. Three-dimensional models for the methyl-truncated enantiomers of CBT-C (3) are shown in FIG. 2

TABLE 1

| Data set | Functional/basis set |
|---|---|
| ORD (589.3 nm, | B3LYP/6-311G++(2d, 2p) |
| ground state) | ωB97XD/6-311G++(2d, 2p) |
| ECD (time- | B3LYP/6-311G++(2d, 2p) |
| dependent) | CAM-B3LYP/6-311G++(2d, 2p) |
| | ωB97XD/6-311G++(2d, 2p) |

As shown in Table 1, functional and basis sets used in ORD and ECD spectral predictions for CBT-C (3) are provided. All calculations were performed for gas phase and implicit MeOH-solvated models.

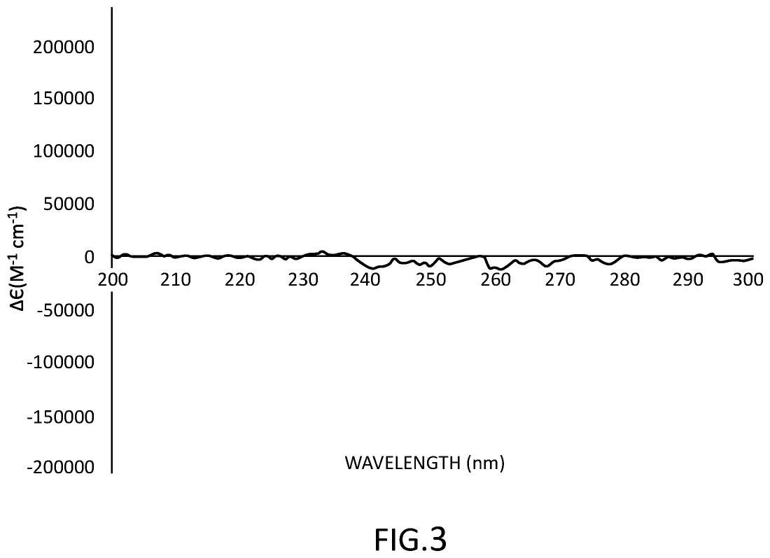

Based on the ECD and TDDFT determinations, ECD data for CBT-C (3) displayed no molar extinction, despite some precedence suggesting the (1R,3R,4S) configuration. A reference standard of CBT-C (3) was obtained, and the experiment was repeated, yielding the same result as shown in FIG. 3. Accordingly, it can be determined that that CBT-C (3) is racemic, and that further analysis with chiral-phase HPLC can be carried out to verify the presence of the two enantiomers.

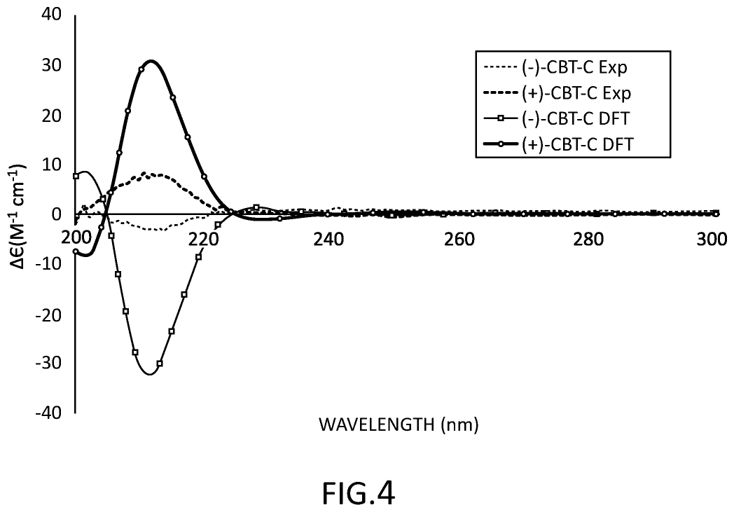

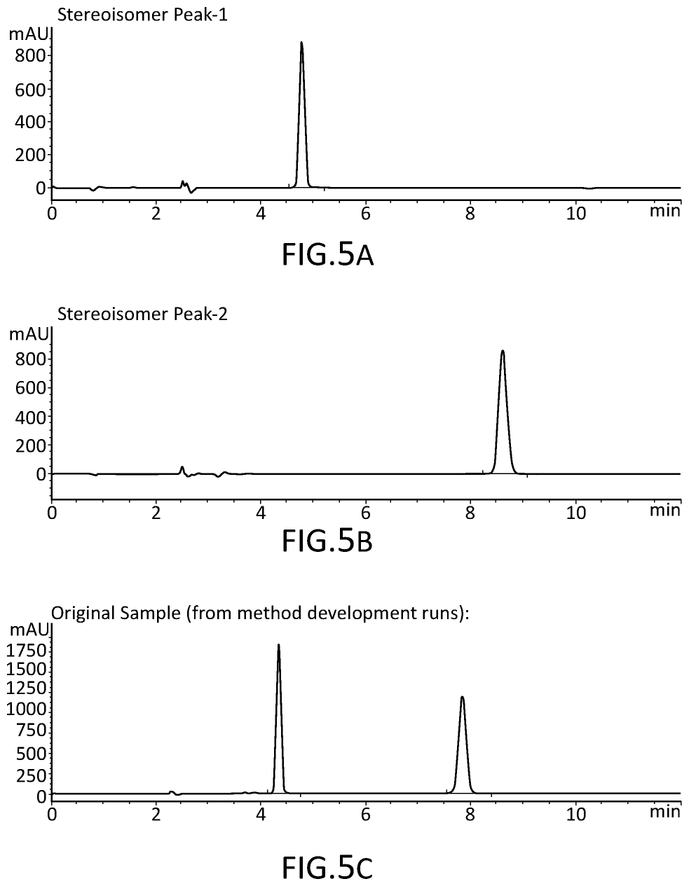

Racemic CBT-C (3) was subjected to HPLC using analytical chiral-phase columns. A CHIRALPAK® IC column efficiently separated the CBT-C (3) enantiomers with equal peak areas (see FIG. 5). Isolation of individual chromatographic peaks provided microgram quantities of each CBT-C (3) enantiomer. ECD experiments were repeated for each enantiomer leading to equal and opposite signals, as is shown in FIG. 4. This separation was successfully scaled up to 1 gram using the same stationary phase (250×30 mm column) and elution with 10% MeOH with 0.1% diethylamine/90% CO₂ at 90 mL/min 100 bar, with UV detection at 220 nm and a 1 mL injection volume of a ˜50 mg/ml solution. Through TDDFT calculations, geometry optimized methyl-truncated structures of CBT-C (3) enantiomers were used to predict ECD spectra for comparison with experimental data. The resulting predicted spectra for each enantiomer are shown below in FIG. 4. Among the various hybrid functionals used, ωB97XD yielded the most accurate results based on wavelength.

According to some embodiments, assigning (−) and (+) to the enantiomers of CBT-C (3) using experimental and DFT-predicted optical rotation data. At 589.3 nm, the experimental specific rotation values were measured to be −4 and +4 degrees/(dm·g/cm⁻³) for (−)-3 and (+)-3, respectively. Through DFT calculations, the specific rotation values were predicted to be −27 and +27 degrees/(dm·g/cm⁻³) for (−)-3 and (+)-3, respectively. As expected, the ωB97XD/6-311G++(2d,2p) functional and basis set provided reasonable predictions for specific rotation based on consistency in magnitude. The mathematical signs of the DFT-predicted specific rotation values provide support for the correct assignment of the enantiomers' absolute configurations.

### II. SFC Purification and Analysis

Preparative supercritical fluid chromatography (SFC) can be carried out to determine characteristics of stereoisomers as a part of a purification process. In one example, the sample was received for preparative SFC chiral resolution of two stereoisomers, and 942 mg was transferred. The preparative SFC separation under the conditions described below in Analytical SFC Conditions and Preparative SFC Conditions yielded 433 mg Peak-1 and 450 mg Peak-2. The purity of the stereoisomers was determined using the analytical SFC conditions which follows and the results are shown in Table 2.

Analytical SFC Conditions

| Instrument | Agilent 1260 SFC | |
|---|---|---|
| Column | Chirapak IC | |
| | 250 × 4.6 mm | |
| Temperature | Ambient | |
| Mobile | 10% MeOH with 0.1% | |
| Phase | diethylamine/90% CO₂ | |
| Flow rate | 2 | mL/min |
| Back pressure | 100 | bar |
| UV wavelength | 220 | nm |

Preparative SFC Conditions

| Column | Chirapak IC | |
|---|---|---|
| | 250 × 30 mm | |
| Temperature | 35° | C. |
| Mobile | 10% MeOH with 0.1% | |
| Phase | diethylmine/90% CO₂ | |
| Flow rate | 90 | mL/min |
| Back pressure | 100 | bar |
| UV wavelength | 220 | nm |

| | |
|---|---|
| Injection volume | 1 mL of ~50mg/mL |

TABLE 2

| ID | % Peak-1 | % Peak-2 |
|---|---|---|
| Stereoisomeric | >99.9% | <0.1% |
| product - Peak-1 | | |
| Stereoisomeric | <0.1% | >99.9% |
| product - Peak-2 | | |

Referring to FIGS. 5A-5C, FIGS. 5A-5C illustrate the analytical SFC chromatograms before and after purification.

### EXAMPLE 2

### I. CBT-C Isolation and Characterization

ECD/VCD/OR experiments. UV measurements and ECD experiments for natural CBT-C (3) and the enantiomerically pure (±)-CBT-Cs ((−)-3, (+)-3) were performed on a Chirascan™ Plus circular dichroism spectrometer in acetonitrile using a 1 mm pathlength cell. Concentrations for UV and ECD experiments were adjusted to provide for A≈0.65 at 214 nm. Degrees were measured in (m°) across a wavelength range of 180-300 nm in increments of 1.0 nm. VCD Experiments were performed on a BioTools ChiralIR-2X™ VCD spectrometer equipped with a DualPEM™ accessory, using a 0.20 mm pathlength cell. All infrared (IR) and VCD spectra were acquired across a range of 930-1650 cm⁻¹; these spectra were acquired with a PEM wave retardation factor of λ/4. Concentrations for VCD experiments were adjusted to c≈50 mg/mL in chloroform-d. All ECD, IR, and VCD sample spectra were baselined and blank subtracted. Optical rotation experiments were performed on a Rudolph Research Analytical Autopol® III Automatic Polarimeter. A 50 mm pathlength cell was used, and concentrations were adjusted to c≈0.25 g/dL.

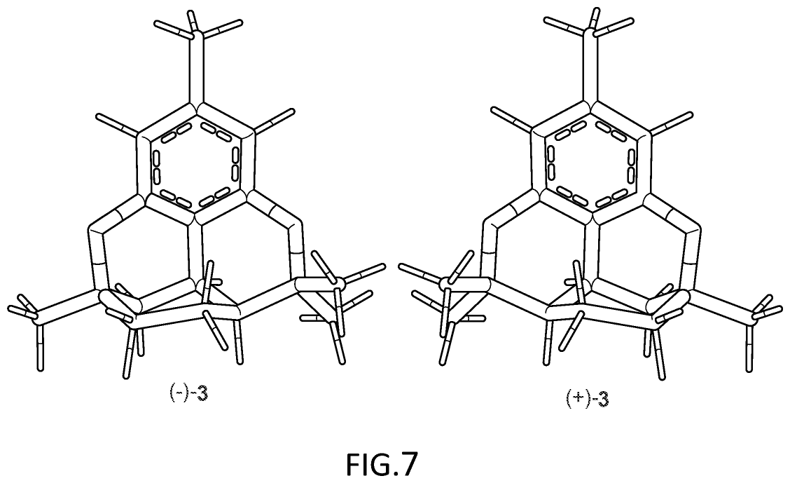

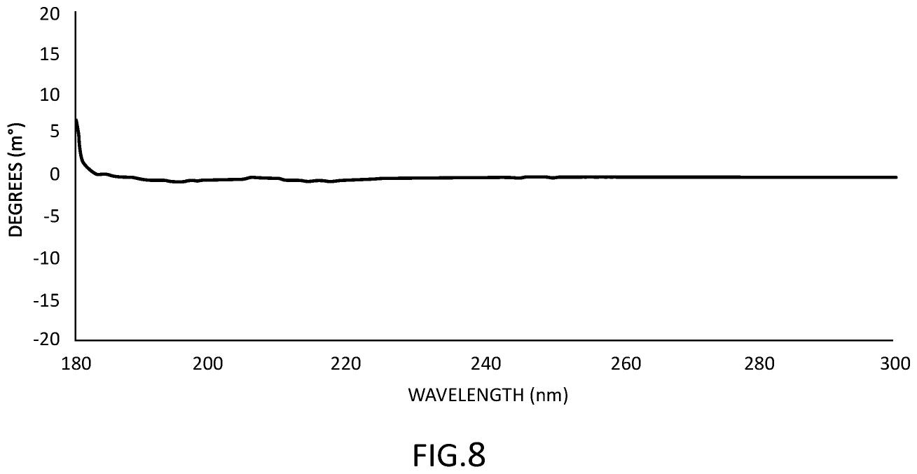

TDDFT calculations. In some instances, the Schrödinger MacroModel software package was used to perform a Mixed Torsional/Low-Mode Sampling (MTLMOD) conformational search using the OPLS4 force field on a methyl-truncated structure of (−)-3, illustrated by FIG. 7. Geometry optimization and frequency calculations were performed on the primary conformer using the B3LYP/6-31G+(d,p) functional and basis set (gas phase). This optimized structure was used to predict optical rotation (OR) and ECD data using the Gaussian software package through TDDFT calculations, further described below with reference to Example 3. The functionals and basis sets used for these calculations are shown below in Table 1. Three-dimensional models for the methyl-truncated enantiomers of CBT-C (3) are shown in FIG. 7.

All TDDFT calculations were repeated using the entire Boltzmann-weighted conformational ensemble of (−)-3 (152 conformers) at the ωB97XD/6-311G++(2d,2p) level of theory using an acetonitrile PCM solvent model to verify that there is negligible influence from the alkyl chain on the predicted ECD spectrum, further described below with reference to Example 3. In some aspects, TDDFT calculations for cannabinoids with olivetol-derived alkyl chains are performed using methyl-truncated structures.

Experimental and TDDFT-predicted UV and ECD data were processed and fitted in SpecDis. All results incorporated the following SpecDis parameters for UV fitting: a band broadening range of 0.1 to 0.4 eV, a shift range of -30 to +30 nm, and a wavelength range of 180 to 300 nm.

DFT Calculations for VCD spectral prediction were performed using the optimal geometry and frequency parameters with Grimme's D3 empirical dispersion correction. All DFT predictions and experimental data were processed based on a maximum IR absorbance of 1. Additionally, DFT calculations were performed on all 152 conformers of (−)-3 to obtain Boltzmann-weighted IR and VCD data in further support of using methyl-truncated molecules for related cannabinoid predictions, further described below.

TABLE 3

| Data set | Functionals | Basis Sets |
|---|---|---|
| ECD (100 excited states, | CAM-B3LYP | 6-311G++(2d, 2p) |
| 180.0-300.0 nm) | ωB97XD | def2-TZVP |
| | | aug-cc-pVTZ |
| OR (589.3 nm) | B3LYP | 6-311G++(2d, 2p) |
| | CAM-B3LYP | def2-TZVP |
| | ωB97XD | aug-cc-pVTZ |
| VCD (930-1650 cm⁻¹) | B3LYP-D3 | 6-31G+(d, p) |

Table 3 provides example functionals and basis sets used in ECD, OR, and VCD predictions for CBT-C (3). Calculations for ECD and OR predictions utilized an implicit (PCM) solvent model for acetonitrile, and calculations for VCD predictions were performed in the gas phase.

### II. Purification and Analysis

An ECD spectrum was acquired after absorbance optimization to facilitate the assignment of CBT-C′s (3) absolute configuration. ECD Data for CBT-C (3) obtained from a CBD extract displayed a near-flat line, as illustrated in FIG. 3, despite precedence suggesting the (1R,3R,4S) configuration. A commercial reference standard of CBT-C (3) was obtained, and the experiment was repeated, yielding the same result thus supporting the racemic nature of CBT-C (3). Chiral-phase HPLC can be used to verify the presence of two enantiomers.

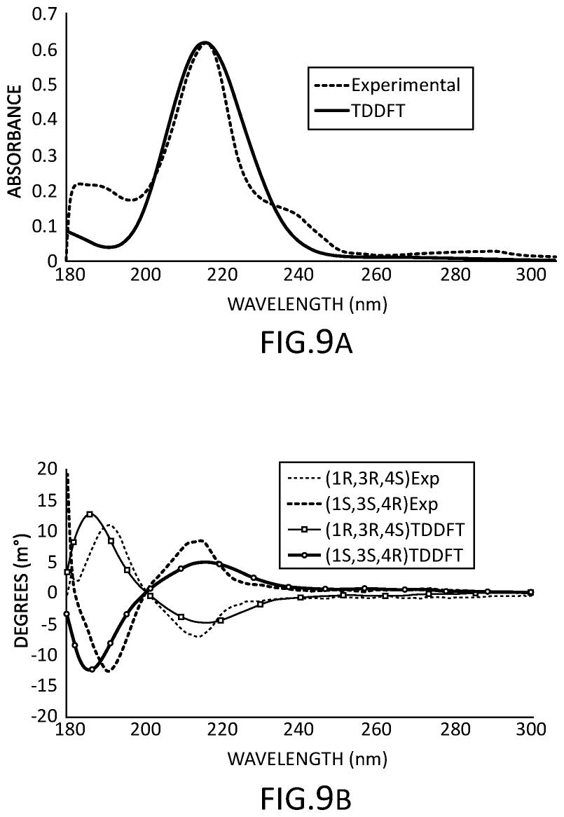

Racemic CBT-C (3) was subjected to HPLC using analytical chiral-phase columns. The CHIRALPAK® IC column efficiently separated the CBT-C (3) enantiomers with equal peak areas. Isolation of individual chromatographic peaks provided microgram quantities of each CBT-C (3) enantiomer. After isolation, ECD experiments were repeated for each enantiomer leading to equal and opposite signals, as illustrated in FIG. 9. This separation was successfully scaled up to 1 gram using the same stationary phase (250×30 mm column) and elution with 10% MeOH with 0.1% diethylamine/90% CO₂ at 90 mL/min 100 bar, with UV detection at 220 nm and a 1 mL injection volume of a ˜50 mg/mL solution.

Through TDDFT calculations, geometry-optimized methyl-truncated structures of CBT-C (3) enantiomers were used to predict ECD spectra for comparison with experimental data. The resulting predicted UV and ECD spectra for each enantiomer are illustrated in FIG. 9. Among the various hybrid functionals used, CAM-B3LYP yielded the most accurate results based on wavelength after spectral fitting in SpecDis.

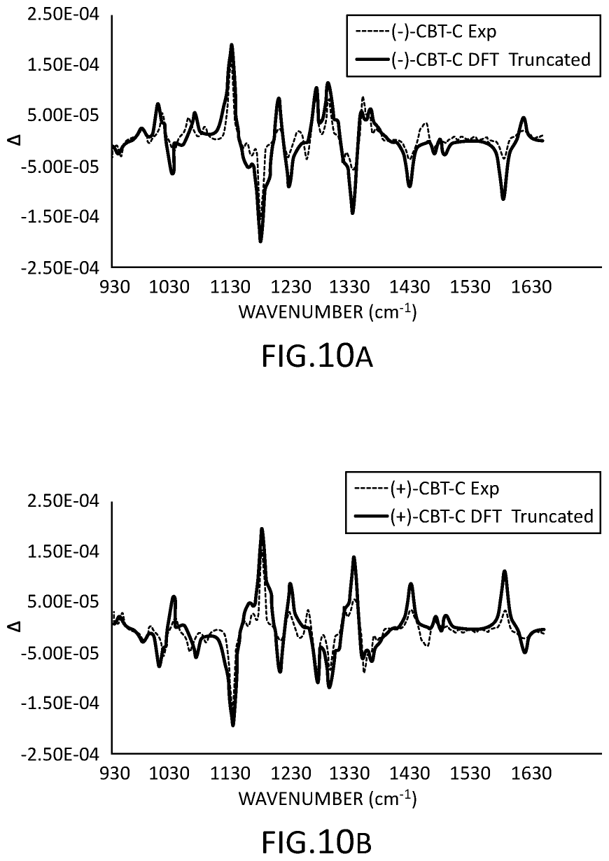

VCD experiments were performed and compared to DFT-predicted data, as illustrated in FIG. 10. The results shown are derived from DFT predictions for the methyl-truncated structure of CBT-C (3). Comparison of the experimental and DFT-predicted spectra clearly support the correct configurational assignments of both enantiomers.

Further, ultimately assigning (−) and (+) to the enantiomers of CBT-C (3) using experimental and TDDFT-predicted optical rotation data can be determined. At 589.3 nm, the experimental specific rotation values were measured to be −15 and +17 degrees/(dm·g/mL) in methanol for configurations (1R,3R,4S) and (1S,3S,4R), respectively. These approximately equivalent magnitudes further support the existence of separate enantiomers. Through TDDFT calculations at the ωB97XD/def2-TZVP level of theory, the specific rotation values were predicted to be −21 and +21 degrees/(dm·g/mL) in methanol for configurations (1R,3R,4S) and (1S,3S,4R), respectively. As expected, both CAM-B3LYP and ωB97XD were excellent functionals for CBT-C (3) ECD and OR predictions, and def2-TZVP was the best-performing basis set overall. It should be noted that while CBT-C (3) has a flexible alkyl chain, OR predictions are known to be more reliable with rigid molecules. Additionally, it is shown that truncation of CBT-C (3) in DFT and TDDFT calculations yields excellent and reliable results, and high accuracy of predicted specific rotations is not necessary for the assignment of (−) and (+) to the individual enantiomers. The mathematical signs of TDDFT-predicted specific rotation values and related cannabinoids (i.e. cis-Δ⁹-THC (1)) provide valuable support for the correct assignment of the enantiomers' absolute configurations. Thus, configuration (1R,3R,4S) is (−)-CBT-C ((−)-3) and configuration (1S,3S,4R) is (+)-CBT-C ((+)-3).

A discussed herein, it is shown that CBT-C (3) isolated from cannabinoid extracts is racemic. The CBT-C (3) enantiomers can be resolved by HPLC using the CHIRALPAK® IC column, where (−)-3 elutes before (+)-3 with significant resolution. According to some aspects, the (−) and (+) enantiomers possess the (1R,3R,4S) and (1S,3S,4R) configurations, respectively. According to some aspects, ECD TDDFT calculations for general cannabinoids are performed at the CAM-B3LYP/def2-TZVP//B3LYP/6-31G+(d,p) level of theory, and OR TDDFT calculations at the ωB97XD/def2-TZVP//B3LYP/6-31G+(d,p) level of theory. Additionally, the implementation of Grimme's D3 empirical dispersion correction in VCD predictions can yield improved results.

### EXAMPLE 3

According to some aspects, a protocol for separation of CBT-C (3) enantiomers is provided. A sample of cannabicitran, isolated from a cannabidiol hemp extract, was used to screen for enantiomer separation on various CHIRALPAK® columns (4.6 μm×250 mm, 5 μm particle size) using hexanes/isopropanol mobile phase on an Agilent 1260 Infinity II HPLC equipped with a diode array detector. A sample of cannabicitran purchased from Cayman Chemical was also analyzed following purification by preparative thin layer chromatography (10:1 hexanes/ether). After optimized conditions were determined, the enantiomers were isolated on analytical scale using 20 μL injections of a ˜5 mg/mL solution. In one example, the separation conditions are: CHIRALPAK® IC, hexanes/isopropanol=92:8, flow rate=1.0 mL/min, 1=280 nm; tR=3.78, 6.65.

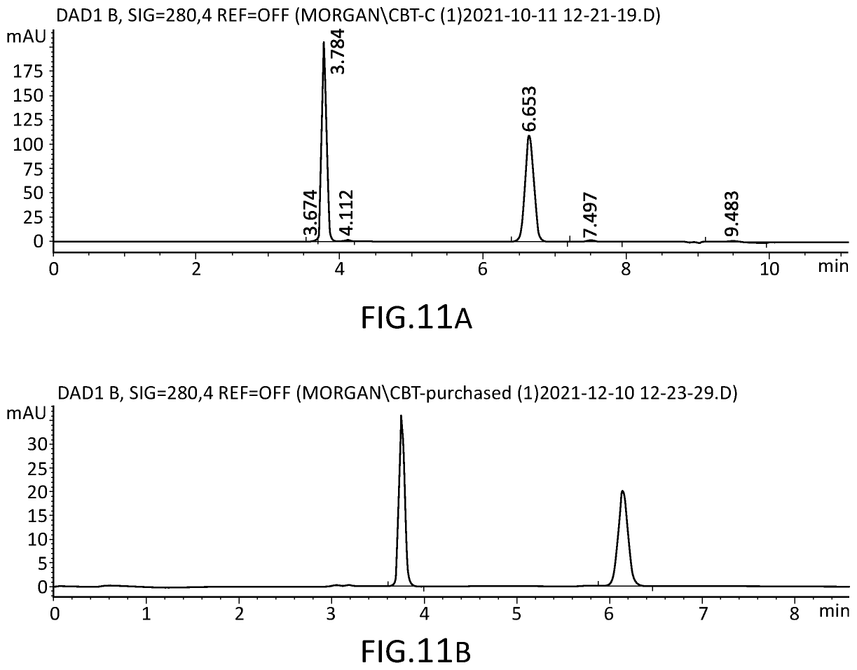

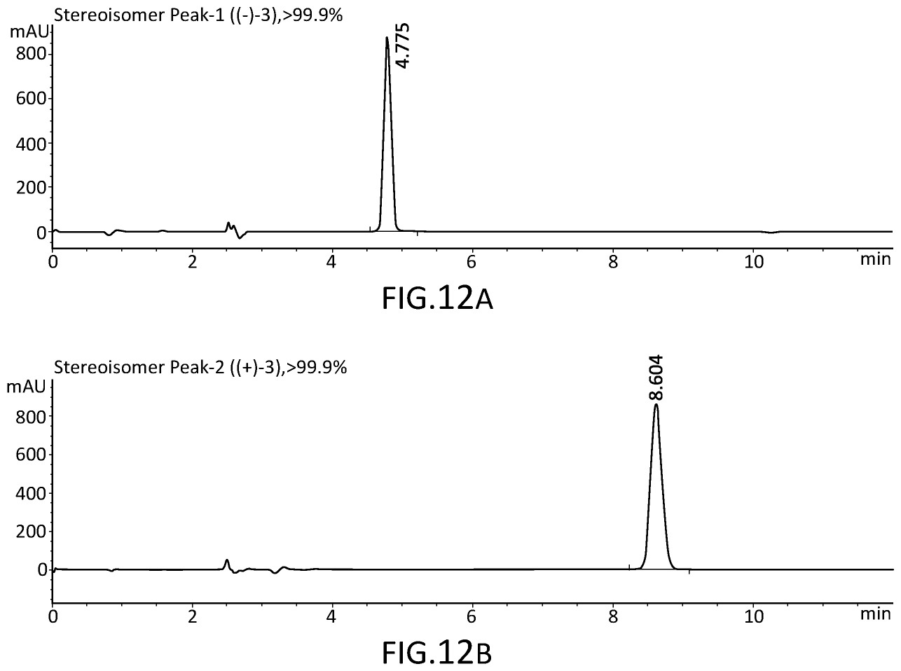

FIG. 11A and FIG. 11B show chromatograms for the cannabicitran from extract as compared to the commercially available cannabicitran, respectively. FIG. 12A and FIG. 12B show analytical SFC chromatograms for the individual CBT-C (3) enantiomers, taken under the following conditions: instrument: Agilent 1260 SFC; column: CHIRALPAK IC 250×4.6 mm; temperature: ambient; mobile phase: 10% MeOH with 0.1% diethylamine/ 90% CO2; flow rate: 2 mL/min; back pressure: 100 bar; UV wavelength: 220 nm.

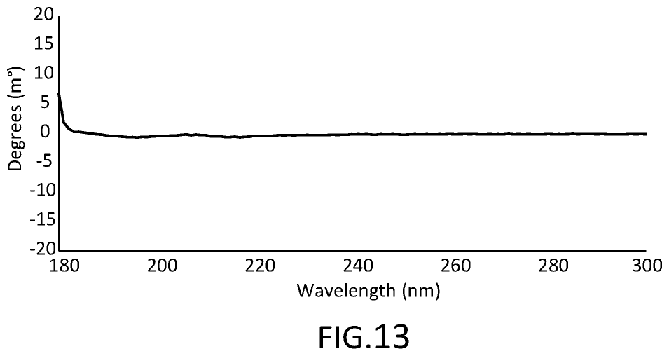

FIG. 13 illustrates experimental data for CBT-C (3) obtained from a CBD extract.

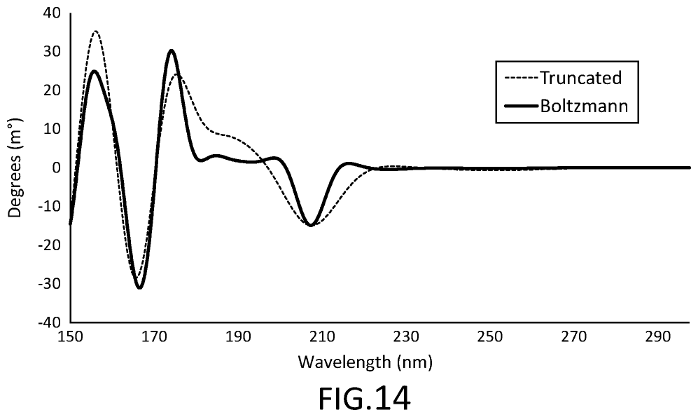

FIG. 14 illustrates a comparison of predicted ECD data for the methyl-truncated and full structures of (−)-3, obtained at the ωB97XD/6-311G++(2d,2p)//B3LYP/6-31G+(d,p) level of theory. Boltzmann weighting was performed using conformers with a population of >2%, and the Boltzmann Magnitude Correction: 1/2.65647971 magnitude.

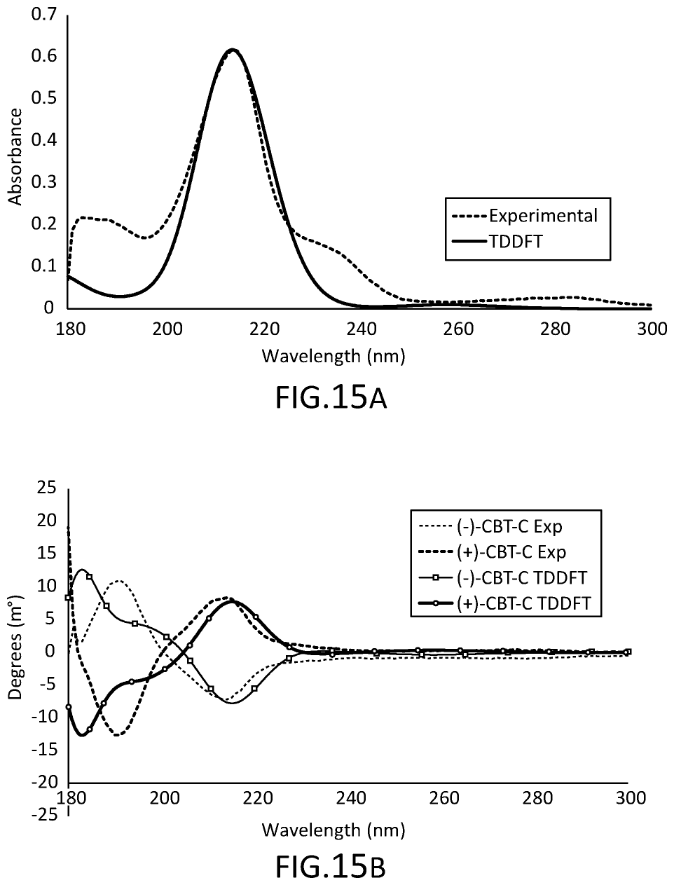

FIG. 15A and FIG. 15B illustrate a comparison of experimental and TDDFT-predicted UV and ECD data obtained at the ωB97XD/6-311G++(2d,2p)//B3LYP/6-31G+(d,p) level of theory. With respect to FIG. 15A and FIG. 15B: sigma=0.29 eV, UV TDDFT Correction: +7.6 nm, 1/104853.916 Absorbance, 0.9669 Similarity Factor, and ECD TDDFT Correction: +7.6 nm, 1/2.07611799 Magnitude, 0.9166 Similarity Factor.

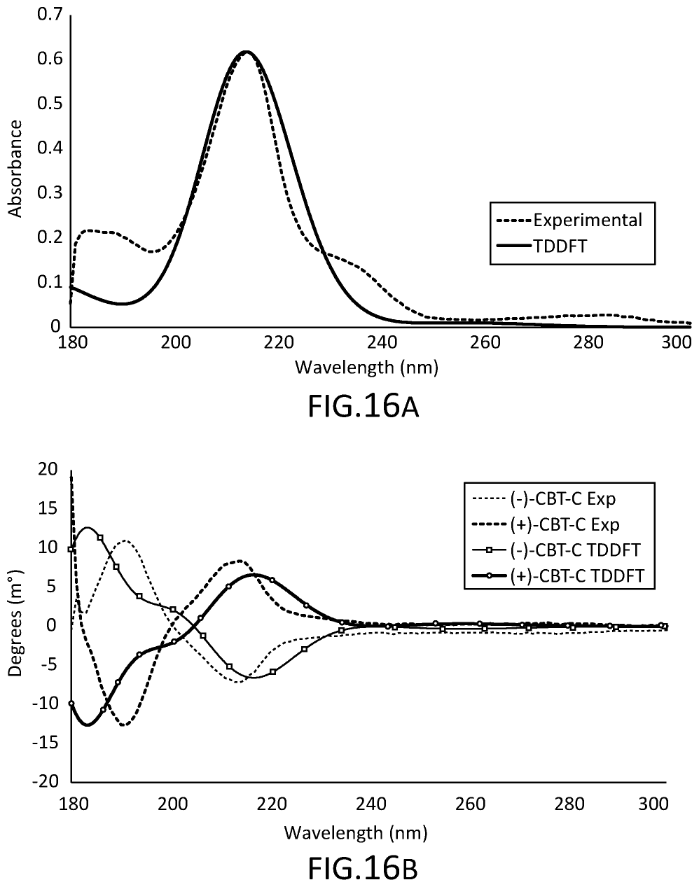

FIG. 16A and FIG. 16B illustrate a comparison of experimental and TDDFT-predicted UV and ECD data obtained at the CAM-B3LYP/6-311G++(2d,2p)//B3LYP/6-31G+(d,p) level of theory. With respect to FIG. 16A and FIG. 16B: sigma=0.34 eV, UV TDDFT Correction: +6.4 nm, 1/88266.4458 Absorbance, 0.9787 Similarity Factor, and ECD TDDFT Correction: +6.4 nm, 1/1.6921551 Magnitude, 0.8958 Similarity Factor.

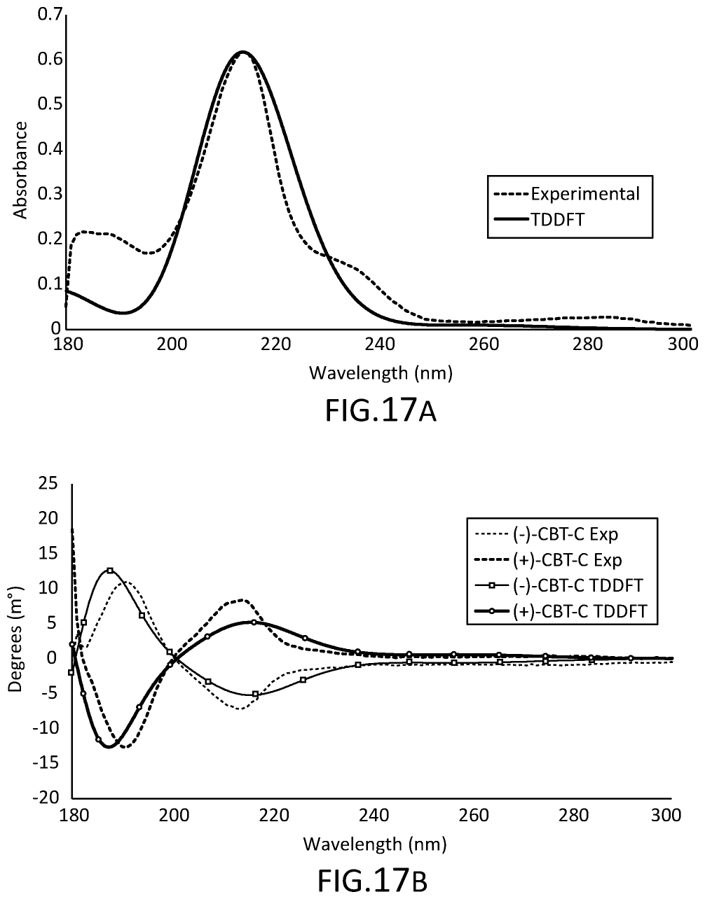

FIG. 17A and FIG. 17B illustrate a comparison of experimental and TDDFT-predicted UV and ECD data obtained at the ωB97XD/def2-TZVP//B3LYP/6-31G+(d,p) level of theory. With respect to FIG. 17A and FIG. 17B: sigma=0.39 eV, UV TDDFT Correction: +10.3 nm, 1/85175.0738 Absorbance, 0.9758 Similarity Factor, and ECD TDDFT Correction: +10.3 nm, 1/1.08998878 Magnitude, 0.9479 Similarity Factor.

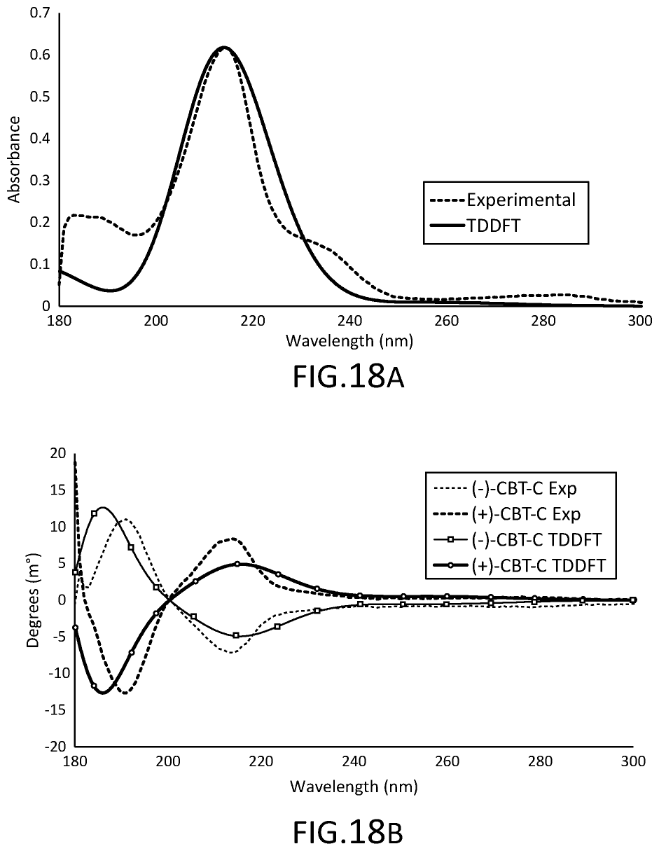

FIG. 18A and FIG. 18B illustrate a comparison of experimental and TDDFT-predicted UV and ECD data obtained at the CAM-B3LYP/def2-TZVP//B3LYP/6-31G+(d,p) level of theory. With respect to FIG. 18A and FIG. 18B: sigma=0.39 eV, UV TDDFT Correction: +9.8 nm, 1/85005.798 Absorbance, 0.9778 Similarity Factor, and ECD TDDFT Correction: +9.8 nm, 1/1.28112434 Magnitude, 0.9490 Similarity Factor (highest).

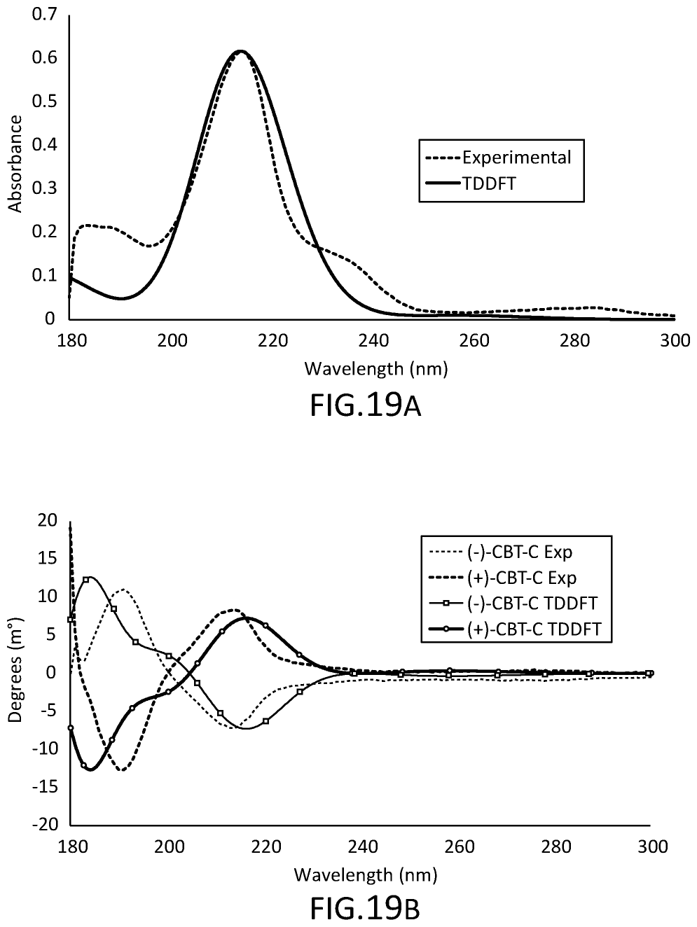

FIG. 19A and FIG. 19B illustrate a comparison of experimental and TDDFT-predicted UV and ECD data obtained at the ωB97XD/aug-cc-pVTZ//B3LYP/6-31G+(d,p) level of theory. With respect to FIG. 19A and FIG. 19B: sigma=0.35 eV, UV TDDFT Correction: +7.6 nm, 1/87086.208 Absorbance, 0.9787 Similarity Factor, and ECD TDDFT Correction: +7.6 nm, 1/1.47438 Magnitude, 0.9072 Similarity Factor.

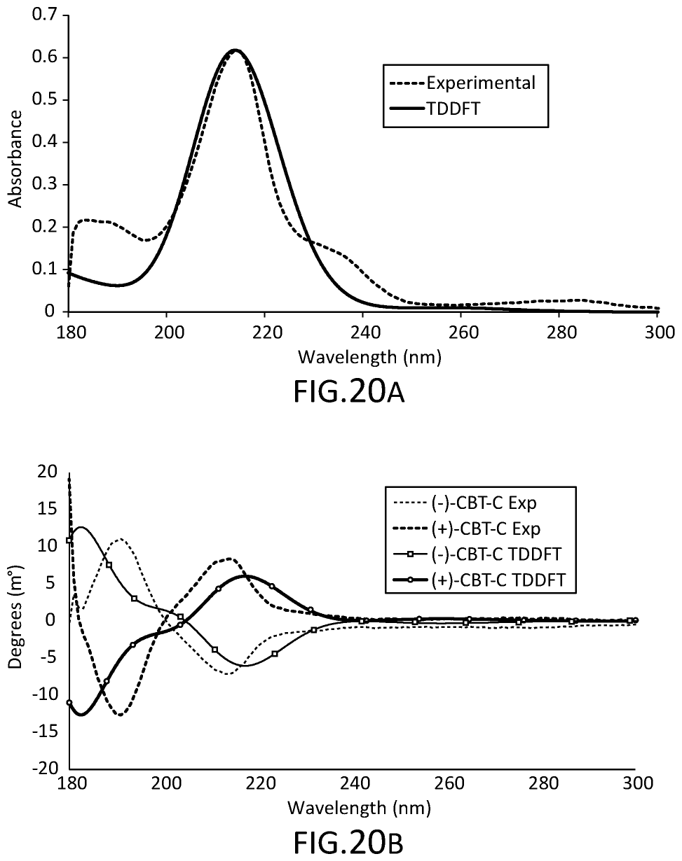

FIG. 20A and FIG. 20B illustrate a comparison of experimental and TDDFT-predicted UV and ECD data obtained at the CAM-B3LYP/aug-cc-pVTZ//B3LYP/6-31G+(d,p) level of theory. With respect to FIG. 20A and FIG. 20B: sigma=0.34 eV, UV TDDFT Correction: +6.2 nm, 1/86324.891 Absorbance, 0.9796 Similarity Factor, and ECD TDDFT Correction: +6.2 nm, 1/1.750285 Magnitude, 0.8810 Similarity Factor.

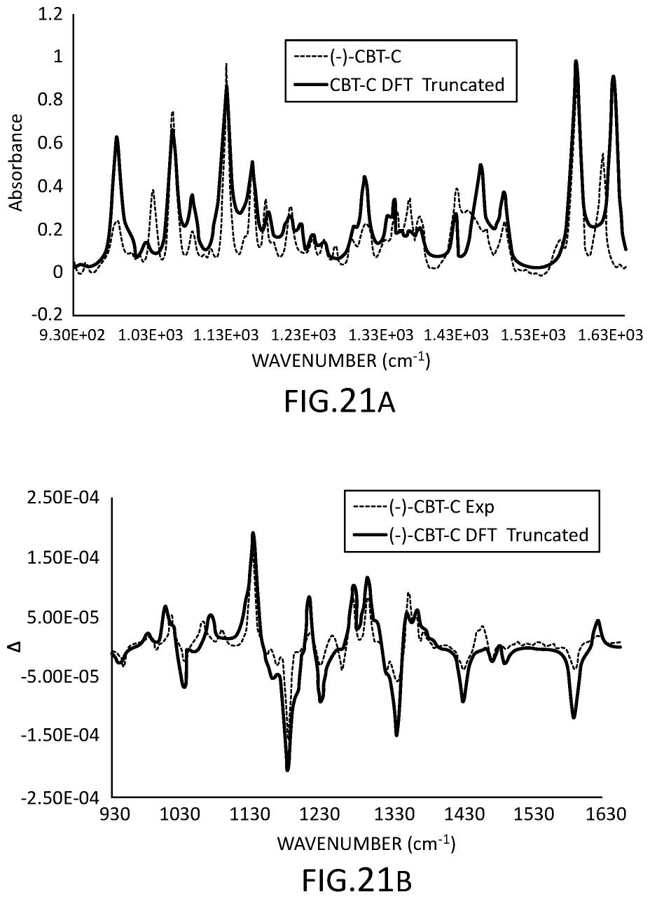

FIG. 21A and FIG. 21B illustrate experimental and DFT-predicted IR and VCD spectra of (−)-3. DFT Predictions are derived from the methyl-truncated structure of (−)-3, obtained at the B3LYP-D3/6-31G+(d,p) level of theory. With respect to FIG. 21A and FIG. 21B, VCD DFT Correction: 0.000202643261545 Magnitude.

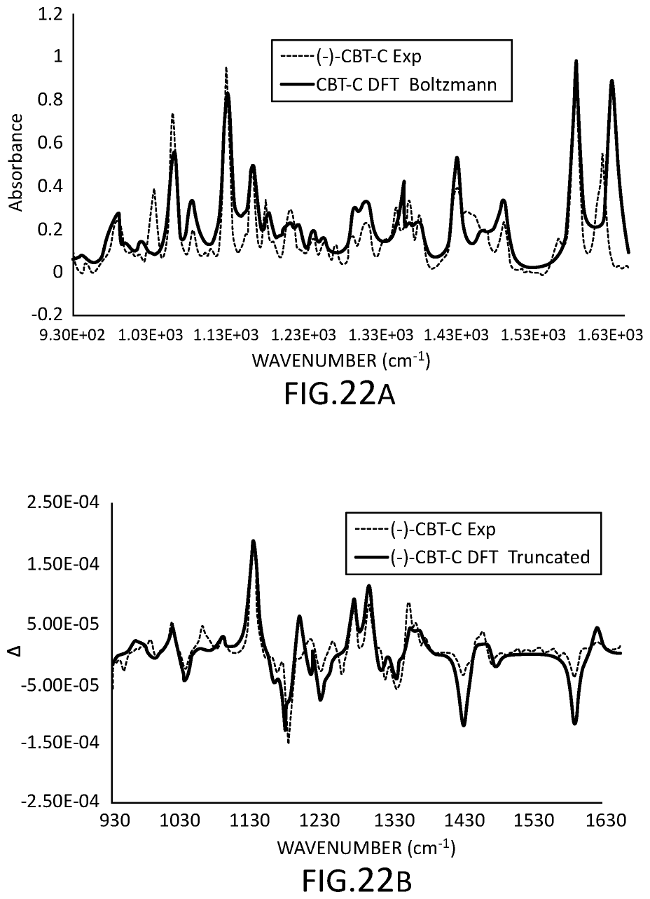

FIG. 22A and FIG. 22B illustrate experimental and DFT-predicted IR and VCD spectra of (−)-3. DFT Predictions are derived from the Boltzmann-weighted conformational ensemble of (−)-3, obtained at the B3LYP-D3/6-31G+(d.p) level of theory. With respect to FIG. 22A and FIG. 22B, VCD DFT Correction: 0.000194713 Magnitude.

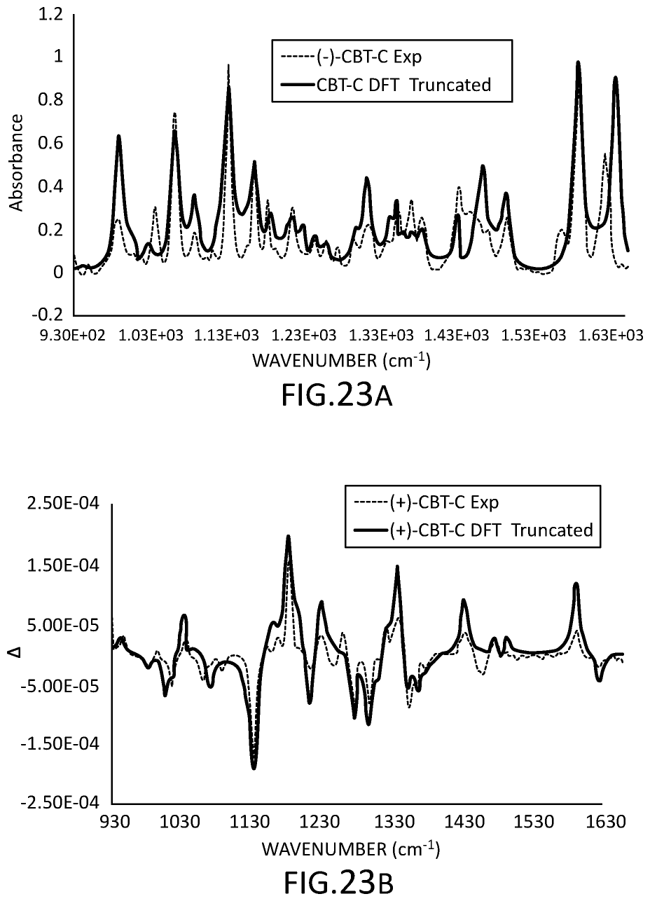

FIG. 23A and FIG. 23B illustrate experimental and DFT-predicted IR and VCD spectra of (+)-3. DFT Predictions are derived from the methyl-truncated structure of (+)-3, obtained at the B3LYP-D3/6-31G+(d,p) level of theory. With respect to FIG. 23A and FIG. 23B, VCD DFT Correction: 0.000202643261545 Magnitude.

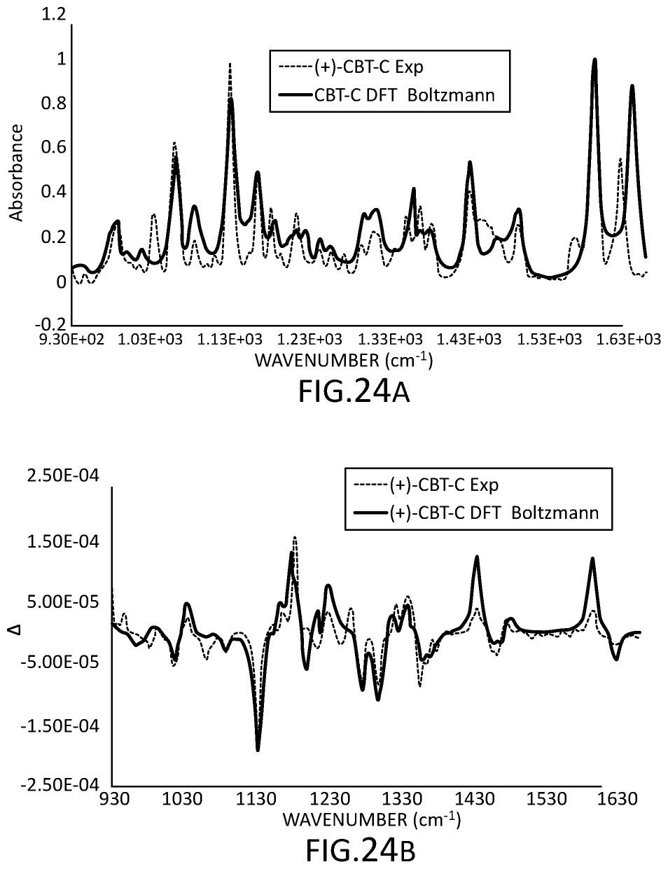

FIG. 24A and FIG. 24B illustrate experimental and DFT-predicted IR and VCD spectra of (+)-3. DFT Predictions are derived from the Boltzmann-weighted conformational ensemble of (+)-3, obtained at the B3LYP-D3/6-31G+(d,p) level of theory. With respect to FIG. 24A and FIG. 24B, VCD DFT Correction: 0.000194713 Magnitude.

Table 4 shows experimental and TDDFT-predicted optical rotation data in methanol for CBT-C (3) at 589.3 nm. All TDDFT calculations utilized a methanol PCM solvent model, and all values displayed in the table below are specific rotations in degrees/(dm·g/mL)). Specific rotation is determined by equation 1, where a=rotation, in degrees, c=concentration, 0.25 g/dL, and l=pathlength, 0.5 dm:

$$Specific rotation {: [ α ]}_{589.3 nm}^{26 ° C .} = \frac{α}{( \frac{c}{100} ) \times l} \qquad ( eq . 1 )$$

TABLE 4

| Methanol | 6-311G++(2d, 2p) | def2-TZPV | aug-cc-pVTZ |
|---|---|---|---|
| ωB97XD | 25.90 | 21.07 | 24.40 |
| | | (Closest) | |
| CAM-B3LYP | 29.86 | 24.20 | 27.69 |
| B3LYP | 41.24 | 34.68 | 39.22 |

| | |
|---|---|
| (−)-CBT-C Experimental | −15.20 |
| (+)-CBT-C Experimental | +16.84 |

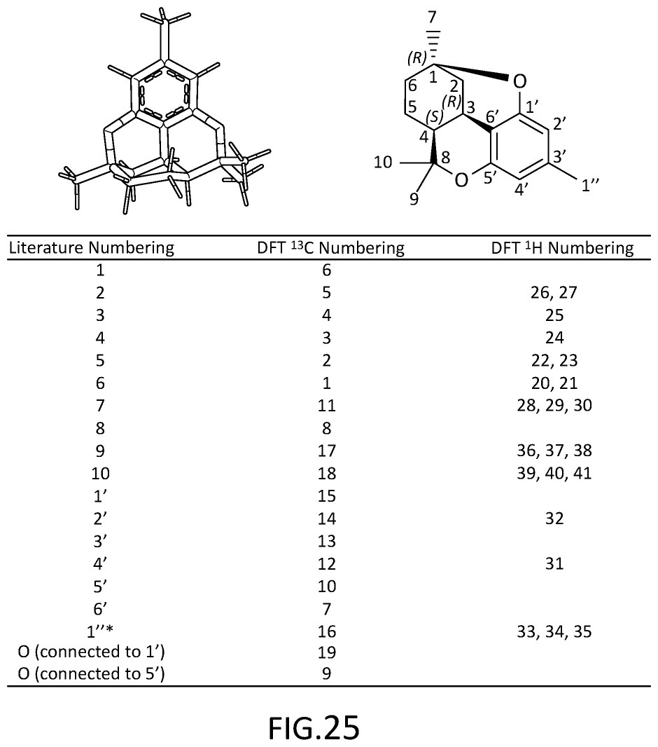

FIG. 25 shows DFT calculation data for the primary conformer (Boltzmann population 99.91%) of the methyl-truncated structure of (−)-3.

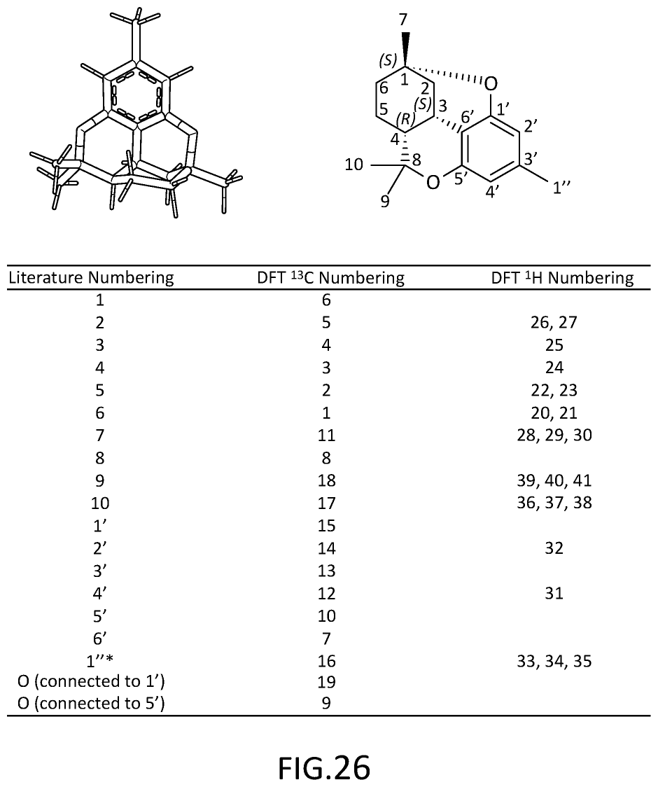

FIG. 26 shows DFT calculation data for the primary conformer (Boltzmann population 99.91%) of the methyl-truncated structure of (+)-3.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention. Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A medicament comprising:
a carrier; and
an amount of cannabicitran (CBT-C).

2. The medicament of claim 1, wherein the CBT-C is an isolated CBT-C (3) racemate.

3. The medicament of claim 2, wherein the CBT-C (3) is enriched in (1R,3R,4S) CBT-C (3) enantiomer.

4. The medicament of claim 2, wherein the CBT-C (3) is enriched in (1S,3S,4R) CBT-C (3) enantiomer.

5. The medicament of claim 1, wherein the CBT-C is (1R,3R,4S) CBT-C (3) enantiomer.

6. The medicament of claim 1, wherein the CBT-C is (1S,3S,4R) CBT-C (3) enantiomer.

7. The medicament of claim 1, wherein the CBT-C is at least one of (+)CBT-C (3) enantiomer and (−)CBT-C (3) enantiomer.

8. A method comprising:
providing a cannabinoid extract; and
isolating at least one cannabicitran (CBT-C (3)) racemate.

9. The method of claim 8, wherein the cannabinoid extract is a CBD extract.

10. The method of claim 9, wherein the cannabinoid extract is a sample of CBT-C (3) obtained from the CBD extract.

11. The method of claim 10, further comprising determining that CBT-C (3) has an ellipticity within 5 degrees of 0 against a wavelength from 180-300 nm.

12. The method of claim 10, wherein two CBT-C (3) enantiomers are separated by a chiral-phase column, and wherein the two separated CBT-C (3) enantiomers have equal peak areas.

13. The method of claim 10, further comprising isolating a first CBT-C (3) enantiomer and a second CBT-C (3) enantiomer based on a chromatographic peak assigned to each of the first enantiomer and the second enantiomer.

14. The method of claim 13, further comprising determining that the first enantiomer and the second enantiomer have equal and opposite ECD signals.

15. The method of claim 13, further comprising assigning a configuration to each of the first enantiomer and the second enantiomer.

16. The method of claim 15, further determining that the first CBT-C (3) enantiomer has a (1R,3R,4S) configuration.

17. The method of claim 15, further determining that the second CBT-C (3) enantiomer has a (1S,3S,4R) configuration.

18. The method of claim 16, further comprising determining that the (1R,3R,4S) CBT-C (3) enantiomer has a specific rotation value between -15 and -25 degrees/(dm·g/ml) in methanol at about 589 nm.

19. The method of claim 17, further comprising determining that the(1S,3S,4R) CBT-C (3) enantiomer has a specific rotation value between 15 and 25 degrees/(dm·g/ml) in methanol at about 589 nm.

20. The method of claim 15, further comprising validating the configuration to each of the first enantiomer and the second enantiomer based on at least one TDDFT determination at a selected level of theory.
